(19) United States
(12) Patent Application Publication
(10) Pub. No.: US 2006/0035971 A1
(43) Pub. Date: Feb. 16, 2006

(54) Blood fluidity improving agent

(75) Inventors: Youichi Arai, Tokyo (JP); Takuya Watanabe, Tokyo (JP); Atsushi Suzuki, Tochigi (JP); Hiroko Joukura, Tochigi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KAO CORPORATION, Tokyo (JP)

(21) Appl. No.: 10/533,660

(22) PCT Filed: Sep. 3, 2003
(86) PCT No.: PCT/JP03/11244

§ 371 Date: May 5, 2005

(30) Foreign Application Priority Data
Nov. 6, 2002 (JP) 2002-322474
Nov. 7, 2002 (JP) 2002-323623

(51) Int. Cl. A61K 31/225; A61K 31/192

(52) U.S. Cl. 514/548; 514/570

(57) ABSTRACT

A blood fluidity-improving agent, a blood circulation promoter or a cerebrovascular disease-improving agent, containing as an active ingredient thereof one or more ingredients selected from the group consisting of chlorogenic acids, caffeic acids, ferulic acids and pharmaceutically acceptable salts of these acids. The present blood fluidity-improving agent, blood circulation promoter or cerebrovascular disease-improving agent is highly safe and can be orally taken over longer periods of time.

### FIELD OF THE INVENTION

The present invention relates to a blood fluidity-improving agent, a blood circulation promoter, and a cerebrovascular disease-improving agent.

### BACKGROUND OF THE INVENTION

The circulation of blood is greatly involved in the roles of supplying human body tissues with oxygen and nutrients while excreting waste products therefrom, thereby performing important functions therein.

Recently, in our life environments, increasingly more people suffer from poor circulation at various parts of their bodies as they get involved increasingly in circumstances where they remain in a fixed posture for extended periods of time, owing to introduction of computers and other information equipment or due to other reasons. A seasonal change also brings about changes in our vital life, for example, it may often invites poor circulation in peripheral circulatory system in a winter season. Further, no one can avoid deterioration of physical functioning owing to his aging, and thus poor circulation due to aging is a great concern as well.

Such poor circulation caused by life environments, seasonal changes, aging and other various factors may threaten the vital activity at various body parts and in many cases result in disorders of mind and body.

Ingredients known heretofore as effective in promoting the blood circulation or improving the blood fluidity which is a factor of blood circulation promotion include: collagen peptide (JP-A-2002-121148); Bidens plants, especially Bidens pilosa or its ingredients (JP-A-2002-205954); γ-linolenic acid as used singly or in combination with a fat-soluble antioxidant (JP-A-2000-302677); dilazep and its acid addition salts (JP-A-1999-92382); hydroxymehylfurfural derivatives (JP-A-1999-228561); estrogen agonists (JP-A-1998-7564); fermented vinegars (JP-A-1998-28567); mulberry leaves, Japanese apricot kernels, Japanese apricot pulps, beefsteak plant leaves, and like materials (JP-A-1998-127253); plasmins and plasminogen activators (JP-A-1996-40931); hyaluronic acids (JP-A-1996-53356); and bilobalides contained in ginkgo leaves (JP-A-1995-53371).

The cerebral apoplexy is a kind of cerebrovascular diseases and is generally classified into cerebral infarction, cerebral hemorrhage and so forth. Statistics reveals that in Japan the number of fatalities due to cerebral apoplexy is so large that this disease ranks third following cancers and ischemic heart diseases that are ranked first and second, respectively.

Risk factors of cerebrovascular diseases may include hypertension, diabetes, hyperlipidemia, smoking, alcohol drinking, obesities and stresses which are factors typically resulting from our daily lifestyle habits and it is apprehended that those risk factors will grow and increase as the society becomes aged.

Conventionally, active ingredients for the prevention of cerebrovascular diseases are available, for example, in the form of calcium antagonists, ACE inhibitors, αβ blockers, etc. that are used as drug medicines, while for this purpose it is also proposed to use glycerophoholipids having fatty acid residues containing a docosahexaenoyl group(s) (JP-A-2000-239168), MCP-1 (Monocyte Chemotactic Protein-1) inhibitors (JP-A-1999-60502), compounds having an anti-endothelin action (JP-A-1998-72363), chitosan (JP-A-1998-182469), activated protein C (JP-A-1995-233087), haptoglobins (JP-A-1994-128173), etc.

### DISCLOSURE OF THE INVENTION

However, as matters stand now, those medicines used for improving the blood fluidity, promoting the blood circulation or improving cerebrovascular diseases are satisfactory as far as their therapeutic efficacies are concerned, but nevertheless patients tend to be heavily burdened with such side effects as hemostatic difficulties, excessive pressure drops during nighttime, low blood pressure, dry coughs, headaches, vertigoes, etc. that are more or less involved in the use of such drug medicines. Meanwhile, those foods or their active ingredients which have been said to be effective in improving the blood fluidity, promoting the blood circulation or improving cerebrovascular diseases are not always satisfactory in respect of their efficacies, and further, it takes a longer time for them to exhibit such effects of improving the blood fluidity, promoting the blood circulation, or improving cerebrovascular diseases.

Accordingly, an object of the present invention is to provide a blood fluidity-improving agent, a blood circulation promoter or a cerebrovascular disease-improving agent which not only work highly effectively to improve the blood fluidity, to promote the blood circulation and to improve cerebrovascular diseases, but also have an excellent safety and thus hardly place any substantial burdens on users in daily ingestion.

With a view to achieving the aforementioned object, the inventors have undertook a series of studies from various aspects to search for certain ingredients which are effective in improving the blood fluidity, promoting the blood circulation or improving cerebrovascular diseases out of ingredients which can be taken in or ingested over a long period of time and are highly safe to use. As a result, they found out that one or more ingredients selected from the group consisting of chlorogenic acids, caffeic acids, ferulic acids and pharmaceutically acceptable salts of these acids can be effectively used as a blood fluidity-improving agent, a blood circulation promoter and a cerebrovascular disease-improving agent. The inventors have also found out that administering one or more ingredients selected from chlorogenic acids, caffeic acids, ferulic acids and pharmaceutically acceptable salts of these acids to persons affected by their lowered functions of peripheral circulation improves their peripheral circulation, so that their cold hands and feet and their body temperature decrease can also be improved. Further, the inventors have found out that the above-described ingredients according to the present invention are substantially free from such side effects as observed in medicines in general and may be easily taken in daily life and thus can be usefully applied in the fields of health foods and medicines.

Specifically, the present invention provides a blood fluidity-improving agent, a blood circulation promoter, a body coldness-improving agent, a body temperature decrease-improving agent or a cerebrovascular disease-improving agent, containing as an active ingredient one or more ingredients selected from the group consisting of chlorogenic acids, caffeic acids, ferulic acids and pharmaceutically acceptable salts of these acids.

The present invention also provides use of one or more ingredients selected from the group consisting of chlorogenic acids, caffeic acids, ferulic acids and pharmaceutically acceptable salts of these acids for the manufacture of a blood fluidity-improving agent, a blood circulation promoter, a body coldness-improving agent, a body temperature decrease-improving agent or a cerebrovascular disease-improving agent.

Further, the present invention provides a method of improving the blood fluidity, a method of promoting blood circulation, a method of improving the body coldness, a method of improving the body temperature decrease, and a method of improving cerebrovascular diseases, including administering an effective dose of one or more ingredients selected from the group consisting of chlorogenic acids, caffeic acids, ferulic acids and pharmaceutically acceptable salts of these acids.

Since the blood fluidity-improving agent, blood circulation promoter, body coldness-improving agent, or body temperature decrease-improving agent of the present invention improves the blood fluidity, blood circulation, body coldness or body temperature decrease, these agents are usefully applied for the prevention and treatment of any poor blood circulation caused by life environments, seasonal changes or aging. Likewise, the cerebrovascular disease-improving agent of the present invention is useful for the prevention and treatment of cerebral infarction, cerebral hemorrhage and cerebral apoplexy. The above-described agents of the present invention are highly safe and can be orally taken over long periods of time and thus can have useful applications in functional foods and in foods for specified health use besides medicines.

### DETAILED DESCRIPTION OF THE INVENTION

The chlorogenic acids, caffeic acids and ferulic acids used in the present invention may be extracted from natural products, especially plants, containing such acids or may be industrially produced by chemical synthesis.

The chlorogenic acids, caffeic acids and ferulic acids according to the present invention are present as stereoisomers and the present invention may employ these acids in the form of either pure isomers or mixtures thereof. Specifically, the chlorogenic acids include 3-caffeylquinic acid, 4-caffeylquinic acid, 5-caffeylquinic acid, 3,4-dicaffeylquinic acid, 3,5-dicaffeylquinic acid, 4,5-dicaffeylquinic acid, 3-ferulylquinic acid, 4-ferulylquinic acid, 5-ferulylquinic acid, and 3-ferulyl-4-caffeylquinic acid (cf “Chemistry and Technology of Coffee Roasting”, by Nakabayashi et al., KOGAKU SHUPPAN CO., LTD., p166-167).

When salified, these chlorogenic acids, caffeic acids and ferulic acids can enhance their water solubility and increased physiological efficacy. As for salts of these acids, any pharmaceutically acceptable salts may be used for the present invention. Basic substances suitably employed to form such salts of the acids described above include, for example, alkali metal hydroxides such as lithium hydroxide, sodium hydroxide, potassium hydroxide, etc.; hydroxides of alkaline earth metals such as magnesium hydroxide, calcium hydroxide, etc.; inorganic alkalis such as ammonium hydroxide, etc.; basic amino acids such as arginine, lysine, hystidine, ornithine, etc.; and organic alkalis such as monoethanolamine, diethanolamine, triethanolamine, etc.; and the hydroxides of alkali metals and alkaline earth metals are particularly preferred. For the present invention, any such salts prepared beforehand may be added to a composition containing the remaining ingredients, or alternatively any chlorogenic acids and salt forming ingredients may be separately added to such a composition so that salts are formed therein.

According to the present invention, preferable natural product extracts, particularly plant extract, containing chlorogenic acids or caffeic acids include, for example, extracts of coffees, cabbages, lettuce, artichokes, tomatoes, eggplants, potatoes, carrots, apples, pears, plums, peaches, apricots, cherries, sunflowers, Jew's marrow, sugarcanes, nandina leaves, blueberries, wheats, etc.

For example, the chlorogenic acids may preferably be extracted from plant matters such as green coffee beans, nandina leaves, unripe apple fruits, etc. More preferably, the chlorogenic acids may be extracted from seeds of Coffee arabica LINNE with a warmed acidic aqueous solution of ascorbic acid or citric acid or with hot water.

More specifically, preferable extracts of green coffee beans include “Flavor Folder” supplied by T. Hasegawa Co., Ltd., Tokyo, Japan and preferable apple extracts include “Applephenon” supplied by THE NIKKA DISTILLING CO., LTD., Tokyo, Japan and preferable sunflower seed extracts include “Heliant” by DAINIPPON INK AND CHEMICALS, INC., Tokyo, Japan.

According to the present invention, preferable natural product extracts containing ferulic acids, particularly preferable plant extracts containing ferulic acids include, for example, extracts of coffees, onions, radishes, lemons, cnidium rhizome, angericae radixes, Pinus, coptis, asafetida, sugarcanes, corns, barleys, wheats, rices, etc., and extracts of rice are particularly preferred. The term “rice” as used herein refers to any raw or dried seeds of Oryza sativa LINNE.

To extract the ferulic acids from a plant, for example, a rice bran oil obtained from a rice bran may be partitioned between aqueous ethanol and hexane phases under alkalescent conditions at room temperature, and then any ferulic acid ester(s) obtained in the aqueous ethanol fraction may be hydrolyzed with sulfuric acid with heat under pressure, followed by refining. The ferulic acids may also be produced by culturing a bacteria (Pseudomonas) in a culture solution containing a clove oil obtained by steam distillation of buds and leaves of Syzygium aromaticum MERRILL et PERRY or in a culture solution containing eugenol prepared by refining such a clove oil, and then subjecting the resultant culture solution to separation and refining.

Further, the ferulic acids may be produced by chemical synthesis, for example, by condensation of vanillin with malonic acid (cf Journal of American Chemical Society, Vol. 74, p5346, 1952).

The above-described ingredients of the present invention may be used in combination of two or more of them. These ingredients are ingested in a total intake ranging preferably from 10 mg to 10 g, more preferably from 35 mg to 5 g and further preferably from 70 mg to 1 g per adult (weighing 60 kg) daily in order to achieve actions to improve the blood fluidity and promote the circulation as intended.

The rheological property of blood is important as a passive factor of blood circulation, especially in microcirculation. For blood capillaries, microarteries and microveins, for example, mechanical properties of blood cells such as erythrocyte deformability and leukocyte adhesivity constitute major factors governing the blood rheology and it is presumed that microcirculation disorders due to any anomalies of such factors constitute causes and symptoms of many diseases.

For measuring effects achieved by the above-described ingredients of the invention to improve the blood fluidity, a number of methods may be employed including: microchannel method (cf “Optically accessible microchannels formed in a single-crystal silicon substrate for studies of blood rheology” by Kikuchi, Y., Sato, K., Ohki H. and Kaneko T. in Microvasc. Res. 44, 226-240 (1992)); laser diffractometry (cf “Modulation of erythrocyte membrane mechanical function by beta-spectrin phosphorylation and dephosphorylation” by Manno S., Takakuwa Y., Nagao K., Mohandas N. in J. Biol. Chem. 270(10), 5659-5665 (1995)); filter method (cf “Regulation of red blood cell filterability by Ca²⁺ influx and cAMP-mediated signaling pathways” by Oonishi T., Sakashita K., Uyesaka N. in Am. J. Physiol. 273(6), C1828-1834 (1997)); and micropipette method (cf “Kinematics of red cell aspiration by fluorescence-imaged microdeformation” by Discher D. E., Mohandas N. in Biophys J.71 (4)1680-1694 (1996)).

Among these, the microchannel method is used commonly.

It is preferred that the present blood fluidity-improving agent be administered to persons whose whole blood transit time ranges from 10 to 1,000 seconds.

To measure effects achieved by the ingredients of the present invention to promote the blood circulation, a number of methods may be employed including: laser Doppler blood flowmeter method (cf “Laser Doppler perfusion imaging of skin blood flow using red and near-infrared sources” by Abbot N. C., Ferrell W. R., Lockhart J. C. and Lowe J. G. in J Invest Dermatol 107 882-886 (1996)); transcutaneous measurement of oxygen partial pressure (cf “Infrapopliteal interventions for limb salvage in diabetic patients” by Hanna G. P., Fujise K., Kjellgren O., Feld S. Fife C., Schroth G., Clanton T., Anderson V., Smalling R. in J. Am. Coll. Cardiol 30 664-669 (1997)); and cold water immersion test (cf “Comparison of alpha-tocopheryl nicotinate and acetate on skin microcirculation” by Kamimura M. in Am. J. Clin. Nutr. 27 1110-1116 (1974)). Among these, the cool water immersion test is commonly used.

It is preferred that the present blood circulation promoter be administered to persons who have a 10 min. or longer skin temperature recovery time of their hands or fingertips as measured in a cold water immersion test, in which the hands or fingertips are immersed in cold water (at 15° C. for 5 minutes) and the time (elapsing for temperature recovery to 25° C.) after removal from the cold water is measured, in other words it is preferred persons requiring a 10 minutes or longer recovery time of finger surface temperature after cold water loading are administered.

Although factors causing vascular disorders include hypertension, diabetes, hyperlipidemia, smoking, alcohol drinking, obesities, stresses, etc., ultimate changes in vascular walls are roughly classified into atherosclerosis and microarterial sclerosis. The atherosclerosis is an arteriosclerosis involving an increase in vascular tunica media and a proliferation of vascular smooth muscle cells, a cholesterin deposition and an infiltration of mononuclear cells and macrophages. Meanwhile, the microarterial sclerosis is found in relatively smaller blood vessels and characterized by necrobiosis and hyalinization of vascular tunica media. An occurrence of such a vascular disorder in the cerebral circulation system represents a cerebrovascular disease, which is one of serious diseases critical to the life.

To evaluate effects achieved by the ingredients of the present invention to improve cerebrovascular diseases in animal experiments, a number of methods have been proposed including those methods based on: stroke-prone spontaneously hypertensive rats (SHRSP) (cf “Importance of genetic factors in hypertensive cerebrovascular lesion; An evidence obtained by successive selective breeding of stroke-prone and resistant SHR” by Yamori Y., Nagaoka A., Okamoto K. in Jpn. circ. J., 38 1095-1100 (1974)); mice (cf “Mouse models of acute subdural hematomas and ischemic lesions” by Sasaki M., Laurence T. D., Teramoto A. in Excerpts from Japan Neurosurgical Society General Convention 59 273 (2000)); rabbits (cf “Low dose nitroglycerin and fasudil hydrochloride combination therapy on rabbit models of subarachnoid hemorrhage” by Isotani E., Itoh Y., Mizuno Y., Ohono K., Hirakawa K. in Cerebrovascular Spasm, 14 313-317 (1999)); and dogs (cf “Efficacy of controlled release nicardipine preparations on dog models of cerebrovascular spasms” by Kawashima A., Kasuya H., Sasahara A., Izawa M., Takakura K., Miyajima M. in Cerebrovascular Spasm, 14 322-325 (1999)).

In the preferred examples of the present invention to be described herein later, the above-described SHRSP method was used to experimentally evaluate effects of the present ingredients on cerebral apoplexy. The SHRSPs are rats of an inbred strain separated from spontaneous hypertensive rats (SHR) by preferential mating. Since aged SHRSPs are susceptible to hypertension and thus cause cerebrovascular diseases, they have been widely used so far as the only animal model that develops cerebral apoplexy spontaneously.

In respect of symptoms, the SHRSPs are closely similar to humans in terms of pathologic conditions, developing both cerebral hemorrhage and cerebral infarction. Pathologically, if arterial necrosis involves ruptures at initial stage, both humans and SHRSPs will undergo cerebral hemorrhage to develop cerebral infarction involving clots.

According to the present invention, one or more ingredients selected from the group consisting of chlorogenic acids, caffeic acids, ferulic acids, and pharmaceutically acceptable salts of these acids may be used effectively as a blood fluidity-improving agent, a blood circulation promoter, a body coldness-improving agent, a body temperature decrease-improving agent or a cerebrovascular disease-improving agent. The blood fluidity-improving agent, blood circulation promoter and cerebrovascular disease-improving agent of the present invention may contain such active ingredients in a quantity ranging preferably from about 0.01 to about 80 wt %, more preferably from about 0.05 to about 60 wt % and particularly preferably from about 0.1 to about 60 wt %.

When using the blood fluidity-improving agent, blood circulation promoter and cerebrovascular disease-improving agent of the present invention as medicines, any pharmaceutically acceptable carrier may be added to such active ingredients to form compositions for oral use or for parenteral use.

Such compositions for oral use include, for example, tablets, granules, subtle granules, pills, powdered drugs, capsules (including hard capsules and soft capsules), lozenges, chewable tablets, food supplements, etc. available as solid medicines or powdered preparations, as appropriate. These preparations cited just above may be provided as foods including supplements for nutritional or dietary purpose.

These preparations may preferably contain the active ingredients of the present invention in a quantity ranging preferably from about 0.1 to about 80 wt % and particularly preferably from about 10 to about 60 wt % in view of effective uptake per day.

When using the blood fluidity-improving agent, blood circulation promoter and cerebrovascular disease-improving agent of the present invention as foods, such foods may be provided in any appropriate forms containing conventional food additives in addition to the present active ingredients, including, besides the aforementioned preparations: beverages, soy sauces, milk, yoghurts, soybean pastes and like liquid, emulsified or paste foods; jellies, gummis and like semisolid foods; and cookies, gums, soybean curds (tofu), etc.

These liquid, emulsified or paste foods, semisolid foods may preferably contain the active ingredients of the present invention in a quantity ranging preferably from about 0.01 to about 50 wt % and particularly preferably from about 0.05 to about 10 wt % in view of effective uptake per day.

According to the present invention, compositions for parenteral use may include preparations for intravenous administration such as injections, suppositories and skin external applications.

The blood fluidity-improving agent, blood circulation promoter and cerebrovascular disease-improving agent of the present invention not only have an excellent safety to permit healthy persons, semi-healthy persons or sick persons to take in daily without causing any problems, but also may be used as food supplements in the form of tablets, granules, etc., various beverages, or various foods, especially foods for specified health use.

### EXAMPLES

Applicable Test 1 Evaluation of Blood Fluidity

i) Experimental Materials and Methodology

(a) Animals Used

Tests were started using stroke-prone spontaneously hypertensive rats (SHRSP/Izm, male) 6 weeks old after acclimating the rats 1 week or longer. The rats were all fed under the conditions of 20 to 26° C. temperature, 40 to 70% humidity and 12 hr. lighting time (from 6 a.m. to 6 p.m.).

(b) Method of Administration and Applied Doses

The rats were administered with samples of the example of the present invention and samples of the comparative example once a day for continuous 28 days from a day when the rats reached 8 weeks of age. For administration, samples were orally administered by forced administration using a disposable polypropylene syringe attached with a metallic oral sonde. For the example, each sample contained a chlorogenic acid (supplied by Sigma Chemical Co., Ltd.) of 50 mg/Kg (body weight)/day, while the comparative example used the equal dose of injection grade water (supplied by Otsuka Pharmaceutical Co., Ltd., Tokyo, Japan) as samples. The preferred example also used the injection grade water as a medium for samples.

For feeds, the rats had been fed freely with a solid feed (CRF-1 supplied by Oriental Yeast Co., Ltd., Tokyo, Japan) for a period from the arrival to grouping of rats and with another solid feed (SP (a feed containing 0.4% common salt) supplied by the same manufacturer as above) for a period succeeding to the grouping. For drinking water, tap water was used in the period from the receipt to grouping of rats and 1% brine in the period after the grouping, respectively, for free uptake.

(c) Test Method

After administering rats with a specified sample for 28 continuous days, blood was sampled from each rat using a VENOJECT II vacuum blood collection tube of 7 mL size (available from Terumo Corporation, Tokyo, Japan) preinfused with 350 μL heparin as an anticoagulant (equivalent to 5% of whole blood). Upon sampling, the blood was agitated quickly to obtain blood samples for measurement. For blood measurement, a cellular microrheological measurement system was used (MC-FAN available from Hitachi Haramachi Electronics Co., Ltd., Ibaragi, Japan). The system incorporates an array of microchannels as a model of blood capillaries to measure the flowing characteristics of blood cells under constant differential pressure. As the model of blood capillaries, a silicon monocrystal basal plate having microchannels of 7 μm in width was used.

First, the time was measured for a 100 μL physiological saline solution to pass through the blood capillary model under differential pressure of 20 cm water column (the measured value to be used later for correction). Then the time was measured (in sec.) for a 100 μL each blood sample to pass through the blood capillary model under the same conditions. The time was measured every 10 μL fraction of sample flow. Each blood sample was microscopically observed as it flowed through the blood capillary model. Using the thus measured transit times as a measure of the blood fluidity, the blood samples were evaluated for their improvement in their fluidity based on the criteria that the shorter transit times represent higher levels of improvement.

ii) Test Results

As is clearly seen from table 1 below showing the test result, the preferred example (a group of rats fed with the chlorogenic acid) exhibit shorter transit times than the comparative example (a group of rats fed with injection grade water), indicating an improvement of the blood fluidity in the preferred example.

TABLE 1

| | |
|---|---|
| Blood volume | Transit time (sec.) |

| passed | Preferred | Comparative |
|---|---|---|
| μL | example | example |
| 10 | 4.87 | 5.46 |
| 20 | 9.67 | 10.59 |
| 30 | 14.31 | 15.64 |
| 40 | 19.34 | 20.73 |
| 50 | 24.59 | 26.10 |
| 60 | 30.03 | 32.24 |
| 70 | 35.90 | 37.93 |
| 80 | 41.65 | 43.70 |
| 90 | 47.58 | 49.90 |
| 100 | 53.38 | 56.34 |

Applicable Test 2 Evaluation of Peripheral Circulation Function by Cold Water Immersion Test (Cooling-Rewarming Test)
i) Experimental Materials and Methodology

As the preferred example of the present invention, subjects of 5 healthy women having a depressed peripheral circulation function each were allowed to drink bottled 125 mL vegetable-fruit mixed juice beverage containing a green coffee beans extract (with 140 mg (0.1 wt %) content as chlorogenic acid) daily one bottle for 6 continuous weeks. As the comparative example, the same 5 subjects, after 3 weeks from the end of the above 6 weeks period of drinking for the preferred example, each were allowed to drink bottled 125 mL vegetable-fruit mixed juice beverage not containing the green coffee beans extract (with 6 mg (0.005 wt %) content as chlorogenic acid) daily one bottle for further 6 continuous weeks. The subjects were evaluated for their peripheral circulation functions through cold water immersion test. After acclimation at 20° C. (50% RH) for 30 minutes, the 5 subjects were tested by immersing their left hands to wrist depth in 15° C. cold water for 5 minutes and the recovery of palmar skin surface temperature of the third digit distal phalanxes of their left hands was measured (using Anritsu HPD-2236 DIGITAL THERMOMETER available from Anritsu Corporation, Kanagawa, Japan).

ii) Test Results

The test result is shown in Table 2 below. The temperatures shown in Table 2 were measured 45 minutes after removal of the left hands from the cold water in the test before and after the subjects had drunk the samples of the preferred example and the comparative example, respectively. As is clearly seen from Table 2, the preferred example exhibits a significant increase in body temperature over the comparative example, indicating that the uptake of the chlorogenic acid has the effect of improving the peripheral circulation function, namely blood circulation. Besides, it was observed in the experiment that the improved blood circulation brought about certain improvements in body coldness and body temperature decrease.

TABLE 2
Temperature recovery in cold water immersion test
(skin temperatures 45 min. after end of immersion)

| | | After 6 weeks |
|---|---|---|
| | Before uptake | uptake |
| Preferred example | 20.5 ± 1.0 | 22.8 ± 2.0* |
| Comparative example | 21.7 ± 1.3 | 22.2 ± 1.4 |

Mean ± standard error (n = 5)
*p < 0.05 vs. before uptake (Wilcoxon signed rank test)

Applicable Test 3 Evaluation of Effects to Improve Symptoms of Body Coldness and Body Temperature Decrease
i) Experimental Materials and Methodology

For 4 male subjects, tablets each containing 19 mg (2 wt %) ferulic acid were prepared as the preferred example of the present invention and otherwise similar tablets not containing (0 wt %) ferulic acid were prepared as the comparative example.

For the test, the subjects each were administered with the sample tablet after rating their symptom levels in the morning and their symptom levels were rated again in the afternoon. The symptom level was rated based on the following 5-step scheme of rating scores and the effect of each sample was evaluated according to the improvement score defined below.

Rating scores

| | |
|---|---|
| 1 | A symptom is felt. |
| 2 | A small symptom is felt. |
| 3 | Neutral. |
| 4 | Symptom is not felt much. |
| 5 | No symptom is felt. |

ii) Test Results

Table 3 shows the test result for the body coldness symptom and Table 4 the result for the symptom of body temperature decrease. For both symptoms, the preferred examples of the present invention showed high improvement scores, revealing their effects of relieving such symptoms.

TABLE 3
Result for body coldness

| | Preferred | Comparative |
|---|---|---|
| | example | example |
| Before uptake | 3.5 | 3.5 |
| After uptake | 4.3 | 3.3 |
| Improvement | 0.8 | −0.2 |
| score | | |

TABLE 4
Results for body temperature decrease

| | Preferred | Comparative |
|---|---|---|
| | example | example |
| Before uptake | 3.5 | 3.8 |
| After uptake | 4 | 3.5 |
| Improvement | 0.5 | −0.3 |
| score | | |

Applicable Test 4 Evaluation of Effects of Improving Cerebrovascular Diseases
i) Experimental Materials and Methodology
(a) Animals Used

Tests were started using stroke-prone spontaneously hypertensive rats (SHRSP/Izm, male) 6 weeks old after acclimating the rats 1 week or longer. The rats were all fed under the conditions of 20 to 26° C. temperature, 40 to 70% humidity and 12 hr. lighting time (from 6 a.m. to 6 p.m.).

(b) Method of Administration and Applied Doses

Rat was administered with samples of the preferred example of the present invention and samples of the comparative example once a day for continuous 49 days from a day when the rats reached 8 weeks of age. For administration, samples were orally administered by forced administration using a disposable polypropylene syringe attached with a metallic oral sonde. For the preferred example, each sample comprised a chlorogenic acid (supplied by Sigma Chemical Co., Ltd.) of 50 mg/Kg (body weight)/day, while the comparative example used the equal dose of injection grade water (supplied by Otsuka Pharmaceutical Co., Ltd., Tokyo, Japan) as samples. The preferred example also used the injection grade water as a medium for samples.

For feeds, the rats were fed freely with a solid feed (CRF-1 supplied by Oriental Yeast Co., Ltd., Tokyo, Japan) for a period from the arrival to grouping of rats and with another solid feed (SP (a feed containing 0.4% common salt) supplied by the same manufacturer as above) for a period succeeding to the grouping. For drinking water, tap water was used in the period from the receipt to grouping of rats and 1% brine in the period after the grouping, respectively, for free uptake.

(c) Test Method

Those animals that survived to the last day of administration were bled to death under pentobarbital for histopathological examination to determine any development of apoplexy. For animals dead during the period of administration, any developments of cerebral apoplexy were examined every time the death occurred.

ii) Test Results

As is clearly seen from table 5 below showing the test result, the preferred example (a group of rats fed with the chlorogenic acid) exhibit definitely suppressed development of cerebral apoplexy than the comparative example (a group of rats fed with injection grade water), indicating effects of improving cerebrovascular diseases in the preferred example.

TABLE 5

| | Number of rats | Cerebral apoplexy |
|---|---|---|
| Groups | used | developed |
| Preferred | 10 | 3 |
| example | | |
| Comparative | 10 | 7 |
| example | | |

### PREFERRED EXAMPLE 1

### As Soft Capsules

| Gelatin | 70.0 (wt %) |
|---|---|
| Glycerin | 22.9 |
| Methyl paraoxybenzoate | 0.15 |
| Propyl paraoxybenzoate | 0.51 |
| Water | 6.44 |

Soft capsule shells each (oval shaped, weighing 150 mg) having the above formulation were filled with a 400 mg soybean oil, 50 mg caffeic acid and 50 mg ferulic acid in a well-known manner to prepare soft capsules.

### PREFERRED EXAMPLE 2

Here, is disclosed an example of the present ingredients used for a beverage.

| Skimmed milk | 3.5 (wt %) |
|---|---|
| Enzymatically decomposed milk casein | 3.5 |
| Fructose | 9.0 |
| Chlorogenic acid | 0.3 |
| Sodium ferulate | 1.0 |
| Citric acid | 0.1 |
| Ascorbic acid | 0.1 |
| Flavor | 0.1 |
| Water | 82.4 |

The beverage of this preferred example having the formulation shown above had high storage stability and a good flavor.

What is claimed is:

1. A blood fluidity-improving agent, comprising as an active ingredient one or more ingredients selected from the group consisting of chlorogenic acids, caffeic acids, ferulic acids and pharmaceutically acceptable salts of these acids.

2. The blood fluidity-improving agent according to claim 1, wherein said one or more ingredients selected from the group consisting of chlorogenic acids, caffeic acids, ferulic acids and pharmaceutically acceptable salts of these acids are contained in a quantity ranging from about 0.01 to about 80 wt %.

3. A blood circulation promoter, comprising as an active ingredient one or more ingredients selected from the group consisting of chlorogenic acids, caffeic acids, and pharmaceutically acceptable salts of these acids.

4. The blood circulation promoter according to claim 2, wherein said one or more ingredients selected from the group consisting of chlorogenic acids, caffeic acids, and pharmaceutically acceptable salts of these acids is contained in a quantity ranging from about 0.01 to about 80 wt %.

5. A body coldness-improving agent, comprising as an active ingredient one or more ingredients selected from the group consisting of chlorogenic acids, caffeic acids, ferulic acids and pharmaceutically acceptable salts of these acids.

6. The body temperature decrease-improving agent, comprising as an active ingredient one or more ingredients selected from the group consisting of chlorogenic acids, caffeic acids, ferulic acids and pharmaceutically acceptable salts of these acids.

7. A cerebrovascular disease-improving agent, comprising as an active ingredient one or more ingredients selected from the group consisting of chlorogenic acids, caffeic acids, and pharmaceutically acceptable salts of these acids.

8. The cerebrovascular disease-improving agent according to claim 7, wherein said one or more ingredients selected from the group consisting of chlorogenic acids, caffeic acids, and pharmaceutically acceptable salts of these acids are contained in a quantity ranging from about 0.01 to about 80 wt %.

9. Use of one or more ingredients selected from the group consisting of chlorogenic acids, caffeic acids, ferulic acids and pharmaceutically acceptable salts of these acids for the manufacture of a blood fluidity-improving agent.

10. The use according to claim 9, wherein said one or more ingredients selected from the group consisting of chlorogenic acids, caffeic acids, ferulic acids and pharmaceutically acceptable salts of these acids are contained in a quantity ranging from about 0.01 to about 80 wt %.

11. Use of one or more ingredients selected from the group consisting of chlorogenic acids, caffeic acids, and pharmaceutically acceptable salts of these acids for the manufacture of a blood circulation promoter.

12. The use according to claim 11, wherein said one or more ingredients selected from the group consisting of chlorogenic acids, caffeic acids, and pharmaceutically acceptable salts of these acids is contained in a quantity ranging from about 0.01 to about 80 wt %.

13. Use of one or more ingredients selected from the group consisting of chlorogenic acids, caffeic acids, ferulic acids and pharmaceutically acceptable salts of these acids for the manufacture of a body coldness-improving agent.

14. Use of one or more ingredients selected from the group consisting of chlorogenic acids, caffeic acids, ferulic acids and pharmaceutically acceptable salts of these acids for the manufacture of a body temperature decrease-improving agent.

15. The use of one or more ingredients selected from the group consisting of chlorogenic acids, caffeic acids, and pharmaceutically acceptable salts of these acids for the manufacture of a cerebrovascular disease-improving agent.

16. The use according to claim 15, wherein said one or more ingredients selected from the group consisting of chlorogenic acids, caffeic acids, and pharmaceutically acceptable salts of these acids is contained in a quantity ranging from about 0.01 to about 80 wt %.

17. A method of improving the blood fluidity, comprising administering an effective dose of one or more ingredients selected from the group consisting of chlorogenic acids, caffeic acids, ferulic acids and pharmaceutically acceptable salts of these acids.

18. The method of improving the blood fluidity according to claim 17, wherein said one or more ingredients selected from the group consisting of chlorogenic acids, caffeic acids, ferulic acids and pharmaceutically acceptable salts of these acids are administered in a daily dose ranging from about 10 mg to about 10 g.

19. A method of promoting the blood circulation, comprising administering an effective dose of one or more ingredients selected from the group consisting of chlorogenic acids, caffeic acids, and pharmaceutically acceptable salts of these acids.

20. The method of promoting the blood circulation according to claim 19, wherein said one or more ingredients selected from the group consisting of chlorogenic acids, caffeic acids, and pharmaceutically acceptable salts of these acids are administered in a daily dose ranging from about 10 mg to about 10 g.

21. A method of improving the body coldness, comprising administering an effective dose of one or more ingredients selected from the group consisting of chlorogenic acids, caffeic acids, ferulic acids and pharmaceutically acceptable salts of these acids.

22. A method of improving the body temperature decrease, comprising administering an effective dose of one or more ingredients selected from the group consisting of chlorogenic acids, caffeic acids, ferulic acids and pharmaceutically acceptable salts of these acids.

23. A method of improving the cerebrovascular disease, comprising administering an effective dose of one or more ingredients selected from the group consisting of chlorogenic acids, caffeic acids, and pharmaceutically acceptable salts of these acids.

24. The method of improving the cerebrovascular disease according to claim 23, wherein said one or more ingredients selected from the group consisting of chlorogenic acids, caffeic acids, and pharmaceutically acceptable salts of these acids are administered in a daily dose ranging from about 10 mg to about 10 g.
